(19) United States
(12) Patent Application Publication
(10) Pub. No.: US 2026/0218901 A1
(43) Pub. Date: Jul. 30, 2026

(54) CARBON MONOXIDE DETECTOR AND SAFETY SWITCH

(71) Applicant: ZeroCo Systems, LLC, Toledo, OH (US)

(72) Inventors: William Kaleb Wilson, Chicago, IL (US); Alfredo Antonio Corrales Reyna, Chicago, IL (US)

(21) Appl. No.: 19/464,084

(22) Filed: Jan. 29, 2026

Related U.S. Application Data
(60) Provisional application No. 63/750,982, filed on Jan. 29, 2025.

(51) Int. Cl. F23N 5/24; G01N 33/00

(52) U.S. Cl. CPC F23N 5/242; G01N 33/0063; F23N 2235/14; F23N 2900/05001

(57) ABSTRACT

A safety system for protecting against a flow of gas includes a gas producing device.

A circuit board controls an operation of the gas producing device. A switch is in communication with the circuit board of the gas producing device. The switch is configured to depower the gas producing device upon a detection of an undesired gas.

### CROSS-REFERENCE TO RELATED APPLICATIONS

This application claims the benefit of U.S. Provisional Application No. 63/750,982, filed on Jan. 29, 2025. The entire disclosure of the above application is hereby incorporated herein by reference.

### FIELD OF THE INVENTION

The invention relates to a safety switch for detecting and militating against a flow of carbon monoxide.

### BACKGROUND OF THE INVENTION

As known, carbon monoxide poisoning is a potentially life threatening situation which occurs by the inhalation of carbon monoxide (CO). Carbon monoxide is an odorless, colorless, tasteless, and non-irritating gas. Because of its difficulty in detection, it is not surprising that how dangerous carbon monoxide can be. Common sources of carbon monoxide include heating and cooking equipment, blocked fireplaces, automobiles left running in enclosed areas, and faulty furnaces. Furnaces, for example, may be installed at various areas within a home such as the basement, the attic or within a closet. Any of these installations could infiltrate the house with carbon monoxide in the event of a malfunction. A mechanical failure, such as a fractured heat exchanger, may force carbon monoxide through the duct-work of the house to create a toxic condition.

Common methods for minimizing carbon monoxide result from two primary mechanisms. The first mechanism relies on knowledge and vigilance. When installing devices that could produce carbon monoxide, it is advisable that such devices are used and installed by professionals in accordance with the manufacture's instructions. Use of portable generators and other portable fuel burning devices are strongly discouraged in an enclosed area such as the home, garage, or other part of a residential space. While such steps are important to minimize the risk of carbon monoxide leaks, the only way to know if the home contains undesirable levels of carbon monoxide is through installation of carbon monoxide detectors.

Typically, carbon monoxide detectors are coupled to an audible alarm in order to alert the homeowner that the levels of carbon monoxide are dangerous and action must be taken. To be effective, therefore, the user must be able to hear the audible alarm and be capable of exiting the contaminated area.

Carbon monoxide detectors, whether battery operated, plug-ins which plug directly into the electrical outlets in a wall, or those detectors directly hardwired into the home's wiring, are known in the art and commercially available. Many of these devices are effective if the homeowner replaces the batteries or keeps the device plugged into the wall outlet. Devices that are hardwired into the user's home may not require the homeowner to care for the device as much as battery operated or plug-ins, but hardwired devices can be expensive, not easily installed, and can fail should the power to the home be disrupted. More importantly, most carbon monoxide detectors that are currently sold use passive means for minimizing the harm from increasing levels of carbon monoxide in an enclosed area. Once the sensors sense a predetermined level of carbon monoxide, the device activates and sounds an alarm. If the alarm signal is heard, it is up to the user to avoid harm by exiting the area. Failure to hear the alarm or failure for the audible signal to trigger results in the homeowner being unaware of the mounting danger. Moreover, some individuals can not remove themselves from the danger, either because they are incapacitated as a result of the carbon monoxide fumes or have pre-existing physically impairments that make it difficult or impossible to escape.

It would therefore be desirable to provide an improved carbon monoxide detector and safety switch directly communicating with the carbon monoxide causing device.

### SUMMARY OF THE INVENTION

In accordance and attuned with the present invention, an improved carbon monoxide detector and safety switch directly communicating with the carbon monoxide causing device has surprisingly been discovered.

According to an embodiment of the disclosure, a safety system for protecting against a flow of gas includes a gas producing device. A circuit board controls an operation of the gas producing device. A switch is in communication with the circuit board of the gas producing device. The switch is configured to depower the gas producing device upon a detection of an undesired gas.

According to another embodiment of the disclosure, a safety system for protecting against a flow of gas from a gas producing device is disclosed. The safety system includes a circuit board configured to provide a powered communication to the gas producing device. The circuit board has a circuit breaker selectively preventing the powered communication to the gas producing device and a switch is configured to detect a concentration level of the flow of gas emitted from the gas producing device, wherein upon detecting the concentration level of the flow of gas, the switch communicates with the circuit breaker of the circuit board to prevent the powered communication to the gas producing device.

### BRIEF DESCRIPTION OF THE DRAWINGS

The above, as well as other objects and advantages of the invention, will become readily apparent to those skilled in the art from reading the following detailed description of an embodiment of the invention when considered in the light of the accompanying drawing which:

FIG. 1 is a flow diagram of a safety system according to an embodiment of the present disclosure; and

FIG. 2 is a schematic illustration of the safety system of FIG. 1.

### DETAILED DESCRIPTION OF THE INVENTION

The following detailed description and appended drawings describe and illustrate various embodiments of the invention. The description and drawings serve to enable one skilled in the art to make and use the invention, and are not intended to limit the scope of the invention in any manner.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls. Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

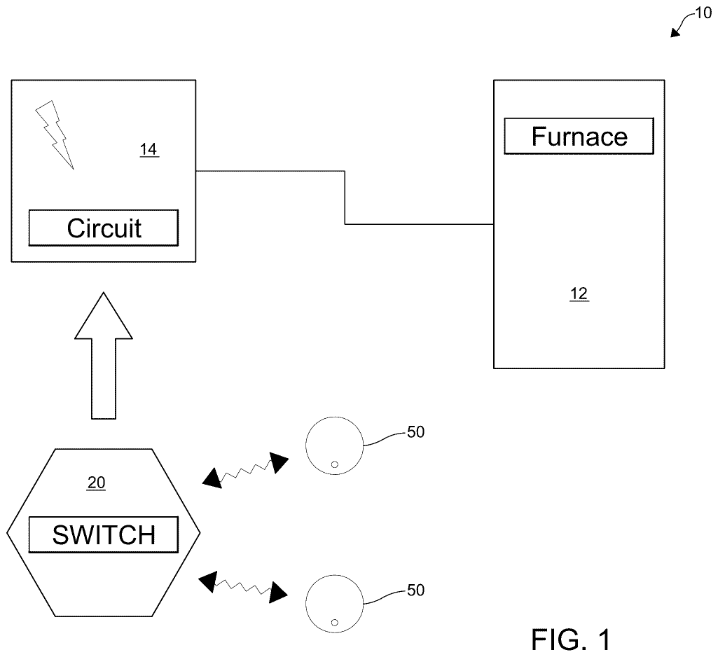

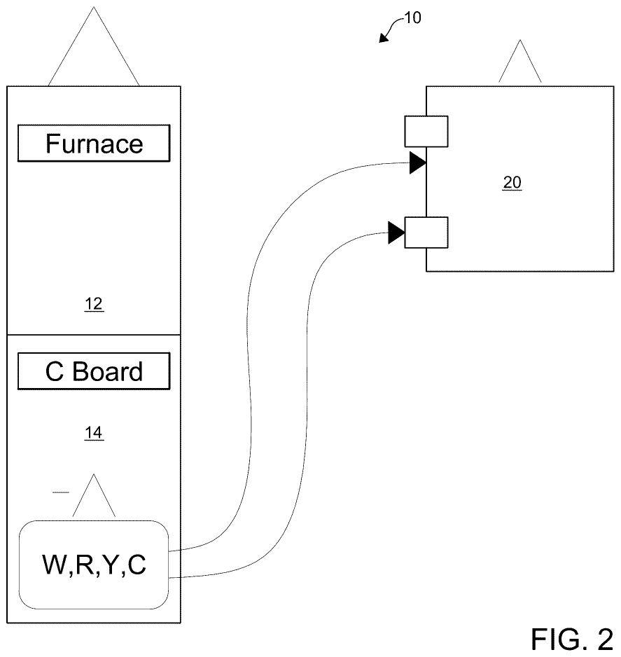

FIGS. 1-2 illustrate a safety system 10 for detecting a gas such as a poisonous or toxic gas, for example. As used herein, the safety system 10 is configured to detect a carbon monoxide (CO) gas. CO gas, as commonly known, is a highly poisonous, colorless, odorless, and tasteless gas produced by the incomplete combustion of fuels like gasoline, wood, and natural gas. CO gas is produced by faulty or poorly ventilated combustion appliances such as, but not limited to, furnaces, water heaters, boilers, ovens, automobile exhaust, especially in enclosed areas, portable generators, wood-burning apparatuses such as chimneys or stoves. However, it is understood the safety system 10 can be configured to detect other harmful or nonharmful gases without departing from the scope of the disclosure.

The safety system 10 includes a gas producing device 12 such as a furnace, for example, a circuit board 14, and a switch 20. The circuit board 14 can be any control board such as printed circuit board (PCB), of the one-sided or two-sided versions, typically incorporated or included with the gas producing device 12 now known or later discovered. However, it is understood the circuit board 14 can be alternative to a PCB such as a solderless breadboard, stripboard, flex circuits, 3D-printed circuits or conductive ink circuit, or any other board or device configured to create electrical pathways, signal pathways, and other pathways between components to operate devices such as the gas producing device. The circuit board 14 can be incorporated within, a housing, a packaging, or a cover for example, the gas producing device 12 or separate from the gas producing device 12, such as mounted or contained externally to packaging of the gas producing device 12.

The switch 20 is in signal communication with the circuit board 14 and the circuit board 14 is in signal communication with the gas producing device 12. The switch 20 can be in wired or wireless communication with the circuit board 14 and the circuit board 14 can be in wired or wireless communication with the gas producing device 12.

The switch 20 can be a safety switch configured to open or close a circuit as a disconnecting means to or otherwise cut off power instantaneously to the gas producing device 12 when reaching a faulty state of operation. For example, the switch 20 is configured to sense carbon monoxide gases at or above 25, 60, 125 PPM, for example. Although the switch 20 can sense any amount of carbon monoxide as desired. The switch 20 can also be configured to detect any gas a desired. For example, when the switch 20 detects the designated level of carbon monoxide, a signal is sent to the circuit board 14 of the gas producing device 12. The signal causes a fuse related to the gas producing device 12 to trip and a gas valve of the gas producing device 12 to close to militate against the flow of the carbon monoxide from exterior of the gas producing device 12. This will permanently shut off the gas producing device 12 completely until it can be professionally examined. However, it is understood the switch 20 can power down the gas producing device 12 in any means necessary depending on the type of the gas producing device 12.

The switch 20 is configured to be coupled to an area in a room of the gas producing device 12 that is within proximity to the gas producing device 12 or susceptible to efficiently detecting concentrations of carbon monoxide. For example, the switch 20 is configured to hang from a ceiling of the room of the gas producing device 12. In another example, the switch 20 is configured to be coupled to a wall of the room of the gas producing device 12. It is understood, the switch 20 can be positioned in any area as desired without departing from the scope of the disclosure as long as the position of the switch 20 is capable of sufficiently detecting carbon monoxide concentration levels.

The safety system 10 is in communication with alerting devices 50. The alerting devices 50 can be one or more smoke and/or carbon monoxide detectors and alarms. However, it is understood the alerting devices 50 can be any alarms, detectors, sensing devices, indicators, or the like without departing from the scope of the disclosure. The safety system 10 can be in direct or indirect wired or wireless communication with the alerting devices 50 such as electrical or signal communication. The safety system 10 is also configured to connect to an application associated device such as a “smart” thermostat, for example, or can be directly associated with the application, wherein the application is downloadable to a computer device such as a smartphone or tablet. The application can then display and notify a user of the computer device of the status of the safety system 10, associated alerting devices 50, and the on/off status of the gas producing device 12.

In application, when one or more of the alerting devices 50 detect the carbon monoxide or other gases, a signal is conveyed to the safety system 10, wherein the switch directly and permanently shuts of the initial source of the carbon monoxide or gas. The computer device can then alert the user of the change of status of the safety system 10 through the associated application.

Advantageously, the switch 20 is configured to directly and permanently shut off the initial source of the carbon monoxide rather than just alerting those within the confines of the gas concentrations.

From the foregoing description, one ordinarily skilled in the art can easily ascertain the essential characteristics of this invention and, without departing from the spirit and scope thereof, can make various changes and modifications to the invention to adapt it to various usages and conditions.

What is claimed is:

1. A safety system for protecting against a flow of gas comprising:
a gas producing device;
a circuit board controlling an operation of the gas producing device; and
a switch in communication with the circuit board of the gas producing device, the switch configured to depower the gas producing device upon a detection of an undesired gas.

2. The safety system of claim 1, wherein the gas producing device is a combustion appliance.

3. The safety system of claim 1, wherein the gas producing device is one of a furnace, a water heater, a boiler, an oven, an automobile exhaust, a portable generator, and a wood-burning apparatus.

4. The safety system of claim 3, wherein the gas producing device is a furnace and the circuit board is incorporated with the gas producing device, and wherein the switch is disposed separately from the gas producing device.

5. The safety system of claim 1, wherein the undesired gas is carbon monoxide.

6. The safety system of claim 5, wherein the switch is configured to detect an amount of carbon monoxide less than about 125 parts per million.

7. The safety system of claim 6, wherein the switch is configured to detect an amount of carbon monoxide less than about 60 parts per million.

8. The safety system of claim 7, wherein the switch is configured to detect an amount of carbon monoxide less than about 25 parts per million.

9. The safety system of claim 1, wherein the switch is coupled to a wall proximate to the gas producing device.

10. The safety system of claim 1, wherein the switch is coupled to a ceiling proximate to the gas producing device.

11. The safety system of claim 1, wherein the switch is in a same room as the gas producing device.

12. The safety system of claim 1, wherein the switch is in communication with at least one alerting device.

13. The safety system of claim 1, wherein the switch is in communication with a plurality of alerting devices.

14. The safety system of claim 13, wherein at least one of the plurality of alerting devices is an alarm.

15. The safety system of claim 13, wherein at least one of the plurality of alerting devices is a carbon monoxide detector.

16. The safety system of claim 13, wherein at least one of the plurality of alerting devices is disposed in a room separate from a room the gas producing device is disposed in.

17. The safety system of claim 13, wherein at least one of the plurality of alerting devices depowers the gas producing device upon a detection of the undesired gas.

18. A safety system for protecting against a flow of gas from a gas producing device comprising:
a circuit board configured to provide a powered communication to the gas producing device, the circuit board having a circuit breaker selectively preventing the powered communication to the gas producing device; and
a switch configured to detect a concentration level of the flow of gas emitted from the gas producing device, wherein upon detecting the concentration level of the flow of gas, the switch communicates with the circuit breaker of the circuit board to prevent the powered communication to the gas producing device.

19. The safety system of claim 18, wherein the circuit breaker is in direct signal communication with a gas valve of the gas producing device.

20. The safety system of claim 18, wherein the safety system is in communication with at least one alerting device.
